(19) United States
(12) Patent Application Publication
(10) Pub. No.: US 2006/0165785 A1
(43) Pub. Date: Jul. 27, 2006

(54) Method of chemically stabilizing pharmaceutical formulations with cholesterol

(76) Inventors: Brian Noga, Durham, NC (US); Mark Lee Sommerville, Durham, NC (US); Michael K. Taylor, Durham, NC (US); Michiel Mary Van Oort, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/329,257

(22) Filed: Jan. 10, 2006

Related U.S. Application Data
(60) Provisional application No. 60/642,633, filed on Jan. 10, 2005.

(51) Int. Cl. A61K 31/56; A61K 31/205; A61K 31/18; A61K 9/20

(52) U.S. Cl. 424/464; 514/171; 514/554; 514/602

(57) ABSTRACT

The invention relates to a method of improving the chemical stability of an active ingredient substance in a particulate formulation in chemically reactive environment comprising associating the active ingredient substance with a chemically stabilising amount of cholesterol to form composite particles comprising said active ingredient substance and cholesterol.

### CROSS-REFERENCE TO RELATED APPLICATIONS

The present application claims priority to U.S. Provisional Application Ser. No. 60/642,633, filed Jan. 10, 2005, the contents of which are incorporated herein by reference.

### FIELD OF THE INVENTION

The present invention relates to the use of cholesterol for the chemical stabilisation of an active ingredient drug substance. More preferably, the present invention also relates to solid pharmaceutical formulations which comprise an active ingredient drug substance, a chemical stabilising amount of cholesterol, and a carrier, wherein the cholesterol acts to chemically stabilize the active ingredient drug substance in a chemically reactive environment.

### BACKGROUND OF THE INVENTION

An important requirement of pharmaceutical formulations is that they should be stable on storage in a range of different conditions. It is known that active ingredient substances can demonstrate instability to one or more of heat, light or moisture and various precautions must be taken in formulating and storing such substances to ensure that the pharmaceutical products remain in an acceptable condition for use over a reasonable period of time, such that they have an adequate shelf-life. Instability of a drug substance may also arise from contact with one or more other components present in a formulation, for example a component present as an excipient.

In some instances, degradation may be exacerbated by the salt form of the active ingredient substance itself reacting with other components in a solid pharmaceutical formulation. In certain instances, the salt moiety of the active ingredient substance, when exposed to atmospheric moisture or other conditions, provides a reactive environment causing degradation of the active substance itself.

In formulating active ingredient substances, it is usual practice in the pharmaceutical art to formulate active ingredient substance with substances known as excipients which may be required as carriers, diluents, fillers, bulking agents, binders etc. Such excipients are often used to give bulk to a pharmaceutical formulation where the active ingredient substance is present in very small quantities. Such substances are generally chemically inert. Over prolonged storage times, or under conditions of extreme heat or humidity, and in the presence of other materials, such inert substances can, however, undergo or participate in chemical degradation reactions.

Carrier substances that are commonly utilised in solid pharmaceutical formulations include reducing sugars, for example lactose, maltose and glucose. Lactose is particularly commonly used. It is generally regarded as an inert excipient.

However, it has been observed that certain active ingredient substances may undergo a chemical reaction in the presence of lactose and other reducing sugars. For example, it was reported by Wirth et al. (J. Pharm. Sci., 1998, 87, 31-39) that fluoxetine hydrochloride (sold under the tradename Prozac®) undergoes degradation when present in solid tablets with a lactose excipient. The degradation was postulated to occur by formation of adducts via the Maillard reaction and a number of early Maillard reaction intermediates were identified. The authors conclude that drug substances which are secondary or primary amines undergo the Maillard reaction with lactose under pharmaceutically relevant conditions.

The present inventors have found that, under accelerated stability conditions, certain inhalable active ingredient substances also undergo degradation in the presence of lactose, possibly also via the Maillard reaction.

Some inhalable dry powder pharmaceuticals are sensitive to moisture, as reported, for example in WO 00/28979 (SkyePharma AG). The presence of moisture was found to interfere with the physical interaction between a carrier and a drug substance and thus with the effectiveness of drug delivery. Improving the dispersion of active particles by reducing forces of cohesion between the particles is also reported in WO 02/43693 (Vectura). Such interference with physical interactions between a carrier and a drug substance is distinct from chemical instability resulting from degradation.

Using excipients, such as cholesterol, in solid pharmaceutical formulations, to improve physical dispersion and release profile of the active components in a inhaled dry powder formulation are described in WO 02/43693. This application also describes the use of excipients to modify the release profile of the active upon delivery to the target site in the lung by creating a barrier between the drug and aqueous fluids in the body. These benefits are attributed to reducing the effect of penetrating moisture on the fine particle fraction (FPF) of an inhaled formulation and to improving resistance of moisture thus prolonging dissolution of the active after delivery to the patient. Excipient use is also disclosed in WO 03/043586 (Advanced Inhalation Research, Inc.), where drug delivery profile modification is attributed to using drug nanoparticles which are agglomerated into low density but respirable sized particles. Such nanoparticles allegedly achieve a slowed or sustained release of drug substance by virtue of the nanometer size of the active particulates in the agglomerates. Cholesterol is mentioned as a potential excipient in such nanoparticle agglomerates. Chemical stability is not addressed.

We have now surprisingly found that chemical interaction of active ingredient substance and reactive environment, e.g. a chemically reactive carrier, may be inhibited or reduced by the presence of cholesterol as a coating, composite material in particles containing an active ingredient substance.

### SUMMARY OF THE INVENTION

In a first aspect thereof the present invention provides a method of inhibiting or reducing chemical interaction between an active ingredient substance and a reactive environment in a solid pharmaceutical formulation, wherein the active ingredient substance is susceptible to chemical interaction with the reactive environment by the addition of cholesterol to a solid pharmaceutical formulation. Reactive environments may be created due to particular salt counter ions to an active drug substance or, as discussed further herein, due to interactions between the active ingredient substance and carriers in a solid pharmaceutical formulation. The chemical stability of the active substance in the formulation during long term storage is thereby improved.

In another aspect the present invention provides a method of inhibiting or reducing chemical interaction between an active ingredient substance and a carrier in a solid pharmaceutical formulation, wherein the active ingredient substance is susceptible to chemical interaction with the carrier, whereby such method comprises associating cholesterol with said active ingredient agent.

The invention also provides a method of inhibiting or reducing chemical degradation of an active ingredient substance in a solid pharmaceutical formulation comprising the active ingredient substance and a carrier, wherein said active ingredient substance is susceptible to chemical interaction with said carrier comprising including cholesterol in said solid pharmaceutical formulation. The chemical stability of the active substance in the formulation during long term storage is thereby improved.

In a further aspect the present invention provides a solid pharmaceutical formulation comprising (a) an active ingredient substance susceptible to chemical interaction, for example, a reactive environment or with a carrier, (b) cholesterol and a (c) a carrier.

Pharmaceutical formulations according to the present invention have greater chemical stability than the corresponding formulations without said ternary agent.

‘Ternary agent’ is used herein to mean a compound used in a formulation in addition to the active ingredient drug substance or substances (the ‘primary’ agent) and a bulk carrier material or materials (the ‘secondary’ agent). In some circumstances more than one ternary agent may be used. Optionally, further substances, possibly named ‘quaternary agents’, may also be present, for example as a lubricant. Any particular ternary or quaternary agent may have more than one effect.

In the present invention the cholesterol ternary agent is capable of reducing or inhibiting interaction between an active ingredient and a carrier in a solid pharmaceutical formulation.

The particles containing cholesterol and active ingredient substance are preferably composite particles. The composite particles contain both cholesterol and the active ingredient substance.

The composite particles may be comprised of micron sized core particles of active ingredient substance which are coated with cholesterol to yield cholesterol coated micron sized composite particles. Preferably the core active ingredient substance particles are less than 10 microns, preferably less than 5 microns, most preferably between 1 and 5 microns, for example, between 1 and 3 microns, such as 2 microns.

Alternatively, the active ingredient substance may be presented as nanometer sized particles which are formed into composite particles comprising cholesterol, such particles are referred to as nanoparticulate dispersions herein. By nanometer sized particles, we mean that the active ingredient substance particles are less than 1000 nanometers, preferably less than 800 nanometers, such as between 750 and 0.01 nanometers, for example, between 500 and 100 nanometers. An advantage associated with this approach is that when crystalline nanometer sized particles are employed and employed into such composite particles, it can be done without effecting the stable crystalline form of the nanoparticles themselves. Thus crystalline structure is maintained in the composite particles. Crystalline materials are generally more stable and therefore may be desirable for such pharmaceutical formulations.

In a still further alternative embodiment, the active ingredient substance may be presented as a molecular dispersion coprecipitated with cholesterol into composite particles comprising both cholesterol and the active ingredient substance.

The resultant composite particles may be of any suitable size for their intended use. For lung inhalation purposes, the composite particles have a mass mean aerodynamic diameter (MMAD) of less than 15 microns, preferably less than 10 microns, most preferably between 1 and 5 microns, for example, between 1 and 3 microns.

These cholesterol/active ingredient substance composite particles are preferably, although not necessarily, formulated with a carrier excipient. The invention finds particular application in formulations in which the carrier is a reducing sugar, for example lactose, maltose or glucose (for example monohydrate glucose or anhydrate glucose). In a preferred embodiment, the carrier is lactose. Alternative carriers include maltodextrin.

The optimal amount of cholesterol present in a particular composition varies depending on the identity of the active ingredient drug substance present, the sizes of the particles and various other factors. In general, cholesterol is preferably present in an amount of from 0.1 to 99%, more preferably between 5 and 40% w/w based on the total weight of the composition. More preferably the cholesterol is present in an amount of from 0.2 to 20% w/w based on the total weight of the composition. Still more preferably, it is preferably present in an amount of from 0.3 to 6% w/w, for example from 0.5 to 4% w/w. (All % values are based on the ratio of the cholesterol to the total weight of the formulation. For example, 5% cholesterol would contain 5% cholesterol and 95% drug substance.)

The active ingredient substance is typically present in an amount of from 0.01% to 50% w/w based on the total weight of the composition. Preferably, the active ingredient substance is present in an amount of from 0.02% to 10% w/w, more preferably in an amount of from 0.03 to 5% w/w, for example from 0.05% to 1% w/w, for example 0.1% w/w.

Preferably, the active ingredient drug substance is one which includes the group Ar—CH(OH)—CH₂—NH—R.

Preferably, the group Ar is selected from 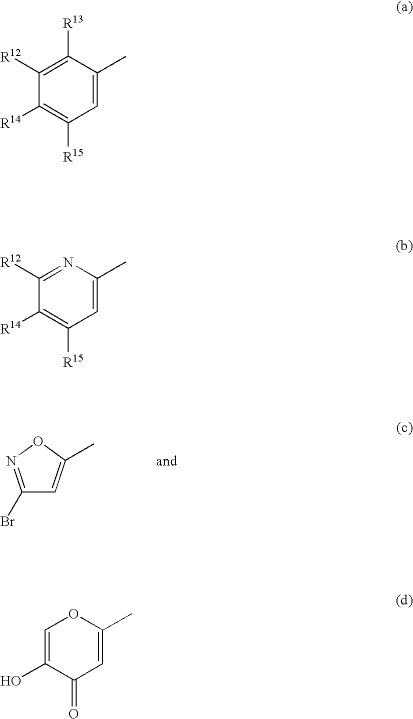
wherein R¹² represents halogen, —(CH₂)_{q}OR¹⁶, —NR¹⁶C(O)R¹⁷, —NR¹⁶SO₂R¹⁷, —SO₂NR¹⁶R¹⁷, —NR¹⁶R¹⁷, —OC(O)R¹⁸ or OC(O)NR¹⁶R¹⁷, and R¹³ represents hydrogen, halogen or C₁₋₄ alkyl;
or R¹² represents —NHR¹⁹ and R¹³ and —NHR¹⁹ together form a 5- or 6- membered heterocyclic ring;
R¹⁴ represents hydrogen, halogen, —OR¹⁶ or —NR¹⁶R¹⁷;
R¹⁵ represents hydrogen, halogen, haloC₁₋₄ alkyl, —OR¹, —NR¹⁶R¹⁷, —OC(O)R¹⁸ or OC(O)NR¹⁶R¹⁷;
R¹⁶ and R¹⁷ each independently represents hydrogen or C₁₋₄ alkyl, or in the groups —NR¹⁶R¹⁷, —SO₂NR¹⁶R¹⁷ and —OC(O)NR¹⁶R¹⁷, R¹⁶ and R¹⁷ independently represent hydrogen or C₁₋₄ alkyl or together with the nitrogen atom to which they are attached form a 5-, 6- or 7- membered nitrogen-containing ring,
R¹⁸ represents an aryl (eg phenyl or naphthyl) group which may be unsubstituted or substituted by one or more substituents selected from halogen, C₁₋₄ alkyl, hydroxy, C₁₋₄ alkoxy or halo C₁₋₄ alkyl; and
q is zero or an integer from 1 to 4;

A physiologically functional derivative of a drug substance, for example of one of the above-mentioned compounds, may also be used in the invention. By the term “physiologically functional derivative” is meant a chemical derivative of a compound of having the same physiological function as the free compound, for example, by being convertible in the body thereto. According to the present invention, examples of physiologically functional derivatives include esters, for example compounds in which a hydroxyl group has been converted to a C₁₋₆alkyl, aryl, aryl C₁₋₆ alkyl, or amino acid ester.

Within the definitions of (a) and (b) above, preferred groups may be selected from the following groups (i) to (xxi): 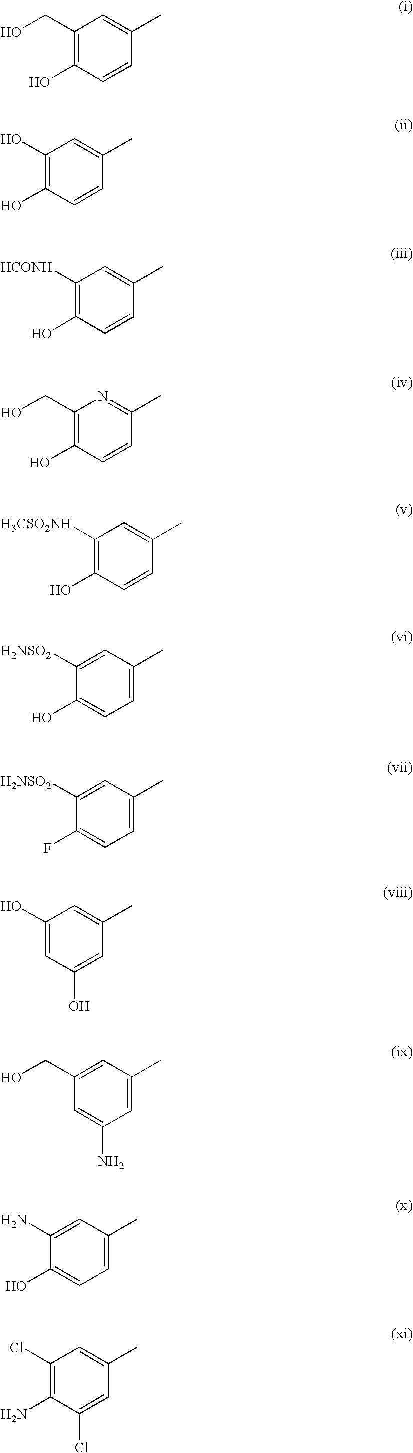 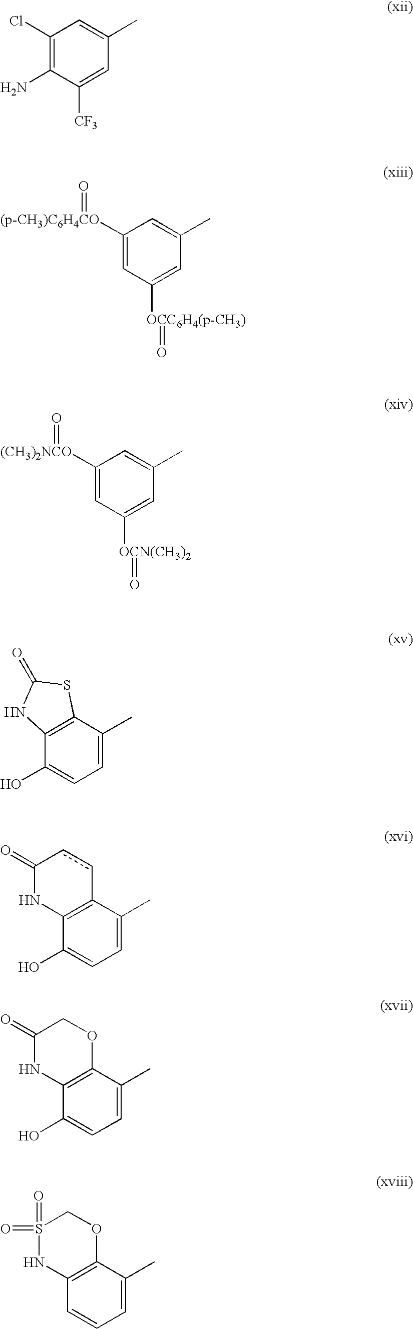 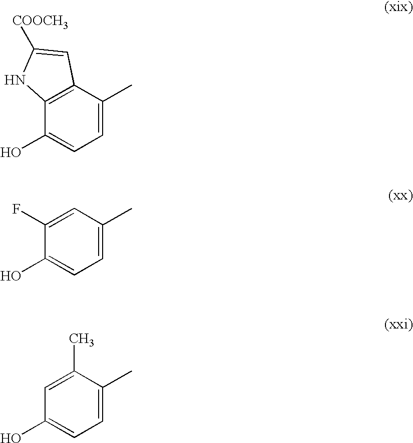
wherein the dotted line in (xvi) and (xix) denotes an optional double bond.

The group R preferably represents a moiety of formula:
-A-B-C-D
Wherein

A may represent (CH2)ₘ wherein m is an integer from 1 to 10;

B may represent a heteroatom, e.g. oxygen;

C may represent (CH₂)ₙ wherein n is an integer from 1 to 10; and

D may represent an aryl group, e.g. an optionally substituted phenyl or pyridyl group.

The active ingredient drug substance may be present as a salt or a solvate. Salts and solvates which are suitable for use in medicine are those wherein the counterion or associated solvent is pharmaceutically acceptable.

Suitable salts for use in the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, phenylacetic, substituted phenyl acetic eg. methoxyphenyl acetic, sulphamic, sulphanilic, succinic, oxalic, fumaric, maleic, malic, glutamic, aspartic, oxaloacetic, methanesulphonic, ethanesulphonic, arylsulponic (for example p-toluenesulphonic, benzenesulphonic, naphthalenesulphonic or naphthalenedisulphonic), salicylic, glutaric, gluconic, tricarballylic, mandelic, cinnamic, substituted cinnamic (for example, methyl, methoxy, halo or phenyl substituted cinnamic, including 4-methyl and 4-methoxycinnamic acid and α-phenyl cinnamic acid (E or Z isomers or a mixture of the two)), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, bezeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexyl amine and N-methyl-D-glucamine.

The active ingredient drug substance is most preferably a selective long-acting β₂-adrenoreceptor agonist. Such compounds have use in the prophylaxis and treatment of a variety of clinical conditions, including diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis).

Other conditions which may be treated include premature labour, depression, congestive heart failure, skin diseases (e.g. inflammatory, allergic, psoriatic, and proliferative skin diseases), conditions where lowering peptic acidity is desirable (e.g. peptic and gastric ulceration) and muscle wasting disease.

Preferred active drug substances for use in the present invention include those described in WO 02/066422, WO 02/070490, WO 02/076933, WO 03/024439, PCT/EP03/02301 and PCT/EP03/04367, the contents of which are incorporated herein by reference as though set out in full herein. For example the drug substance may be 3-(4-{[6-({(2R)-2-hydroxy-2-[4-hydroxy-3-(hydroxymethyl)phenyl]ethyl}amino) hexyl]oxy}-butyl)benzene-sulfonamide, for example as its cinnamate salt. The cinnamate salt of this compound is referred to herein as GW597901 M.

Formulations to which the present invention may be applied include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier and the ternary agent as well as any other accessory ingredients. In general the formulations are prepared by uniformly and intimately bringing into association the active ingredient, lactose, ternary agent and any other accessory ingredients, and then, if necessary, shaping the product into the desired formulation.

Formulations of the present invention suitable for oral administration may be presented as discrete units such as capsules, cachets or tablets each containing a predetermined amount of the active ingredient; as a powder or granules. The active ingredient drug substance may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include sterile powders, granules and tablets intended for dissolution immediately prior to administration. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose an acacia.

The invention finds particular application in dry powder compositions for topical delivery to the lung by inhalation.

Dry powder compositions for topical delivery to the lung by inhalation may, for example, be presented in capsules and cartridges of for example gelatine, or blisters of for example laminated aluminium foil, for use in an inhaler or insufflator. Packaging of the formulation may be suitable for unit dose or multi-dose delivery. In the case of multi-dose delivery, the formulation can be pre-metered (eg as in Diskus, see GB 2242134, U.S. Pat. Nos. 5,837,360, 5,590,645, 5,860,419, 6,032,666, 6,378,519 and 6,536,427 or Diskhaler, see GB 2178965, 2129691 and 2169265, U.S. Pat. Nos. 4,811,731, 5,035,237, 4,627,432 and 4,778,054) or metered in use (e.g. as in Turbuhaler, see EP 69715, U.S. Pat. No. 4524769). An example of a unit-dose device is Rotahaler (see GB 2064336, U.S. Pat. No. 4,353,365). The Diskus inhalation device comprises an elongate strip formed from a base sheet having a plurality of recesses spaced along its length and a lid sheet hermetically but peelably sealed thereto to define a plurality of containers, each container having therein an inhalable formulation containing an active compound. Preferably, the strip is sufficiently flexible to be wound into a roll.

Medicaments for administration by inhalation desirably have a controlled particle size. The optimum particle size for inhalation into the bronchial system of the composite particles is usually 1-10 μm, preferably 2-5 μm. Particles having a size above 20 μm are generally too large when inhaled to reach the small airways. To achieve these particle sizes the particles of the active ingredient substance as produced may be size reduced by conventional means eg by micronisation. The desired fraction may be separated out by air classification or sieving. Preferably, the particles will be crystalline. In general, the particle size of the carrier, for example lactose, will be much greater than the drug substance within the present invention. It may also be desirable for other agents other than the active drug substance to have a larger particle size than the active drug substance. When the carrier is lactose it will typically be present as milled lactose, for example with a MMD of 60-90 μm and with not more than 15% having a particle diameter of less than 15 μm.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds and pharmaceutical formulations according to the invention may be used in combination with or include one or more other therapeutic agents, for example a beta-agonist may be used in combination with one or more other therapeutic agents selected from anti-inflammatory agents (for example a corticosteroid, or an NSAID,) anticholinergic agents (particularly an M₁, M₂, M₁/M₂ or M₃ receptor antagonist), other β₂-adrenoreceptor agonists, antiinfective agents (e.g. antibiotics, antivirals), or antihistamines.

Suitable corticosteroids include methyl prednisolone, prednisolone, dexamethasone, fluticasone propionate, 6α9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors of cytokine synthesis.

Suitable anticholinergic agents are those compounds that act as antagonists at the muscarinic receptor, in particular those compounds which are antagonists of the M₁ and M₃ receptors. Exemplary compounds include the alkaloids of the belladonna plants as illustrated by the likes of atropine, scopolamine, homatropine, hyoscyamine; these compounds are normally administered as a salt, being tertiary amines.

Preferred anticholinergics include ipratropium (e.g. as the bromide), sold under the name Atrovent, oxitropium (e.g. as the bromide) and tiotropium (e.g. as the bromide) (CAS-139404-48-1).

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. Examples of preferred anti-histamines include methapyrilene and loratadine.

The invention further provides the use of an inhalable solid pharmaceutical formulation according to the invention for the manufacture of a medicament for the treatment of diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis). The invention also provides a method for treating asthma, chronic obstructive pulmonary diseases (COPD), chronic or wheezy bronchitis, emphysema, respiratory tract infection upper respiratory tract, or rhinitis, including seasonal and allergic rhinitiscomprising administering to a patient in need thereof an inhalable solid pharmaceutical formulation according to the invention.

In a further aspect, the invention provides a method of preparing a solid pharmaceutical preparation comprising combining in one or more steps: (a) an active ingredient substance susceptible to interaction with the environment, and or a carrier, (b) cholesterol, and optionally (c) a carrier.

### DETAILED DESCRIPTION

### EXAMPLES

In the following examples, the active ingredient substance was salmeterol xinafoate (“SX”), a long acting beta agonist. SX may be produced by the methods known in the art, for example as disclosed in U.S. Patent No. 4,992,474, the teachings of which are incorporated herein by reference.

### Preparation of Composite Particles

### Example 1

### Preparation of Micronized Input Material

SX may be micronized by any known process. For example, an amount of SX was micronized by Trost jet micronizer mill at a slow feed rate, and removed until all material was micronized.

Alternative methods to reduce the particle size of the drug substance to 1-5 μm are known by those skilled in the art.

### Example 2

### Preparation of Composite Particles with Micronized Core Particles.

Approximately 5g of cholesterol was placed into a 500 mL beaker. Approximately 250 mL of propyl acetate was added and stirred to completely dissolve the cholesterol, to yield a clear cholesterol solution. Approximately 5 g of micronized SX, prepared by the method of Example 1, was added to this cholesterol solution to yield a SX suspension. The SX suspension was stirred for 15 minutes. The suspension was sonicated for 1 minute to break any residual agglomerates. The sonicated suspension was fed through the pumping system into a Niro Mobile Minor spray dryer (Niro, Copenhagen, Denmark) using the following process parameters:
Inlet Temperature—50 deg. C.
Final Outlet Temperature—35 deg. C.
Nozzle Atomization Pressure—2 bar
Feedstock feedrate—approx 22.5 mL/min
Process Gas Flow rate: 78 kg/hr
Watson Marlow Solution Pump 505 L
   to produce cholesterol coated SX composite particles. Although this preparation was for a 50% cholesterol particle, one could modify as desire, e.g., 0.5 g chol/9.5 gSX for 5%, 1.5 g chol/8.5 g SX for 15%. 2.5 g chol/7.5 g SX for 25%.

### Example 3

As will be appreciated by those of ordinary skill in the art, nanometer sized particles of may be prepared/produced using a variety of known technologies, e.g., nanomilling.

A particulate dispersion comprised of nanomilled SX input material and cholesterol are spray dried from a feedstock suspension, in this case to produce a 50% cholesterol formulation.

Approximately 5 g of cholesterol was added into a 500 mL beaker. Approximately 200 mL of propyl acetate was added to the beaker and stirred until the cholesterol was fully dissolved forming a clear solution. Approximately 50 mL of the 10%w/v suspension the of nanomilled SX in n-propyl acetate, prepared by the method of Example 3, was added to the cholesterol solution. The resulting suspension (4% w/w solids for a 50% cholesterol formulation) was stirred for 15 minutes prior to spray drying.

This feedstock suspension was spray dried in a suitable spray dryer (such as a Niro Mobile Minor) using the following process parameters:
Inlet Temperature—50 deg. C.
Final Outlet Temperature—35 deg. C.
Nozzle Atomization Pressure—2 bar
Feedstock feedrate—approx 22.5 mL/min
Process Gas Flow rate: 78 kg/hr
Watson Marlow Solution Pump 505 L

To yield composite particles comprising a nanometer sized active ingredient substance dispersed in a cholesterol matrix.

As will be appreciated by those skilled in the art, this may be modified to produce other formulation concentrations. For example, a 5% cholesterol would require 0.5 g cholesterol in 155 ml propyl acetate followed by the addition of 95 ml of the 10% w/v suspension (145/105 for 9% w/v).

### Example 4

### Preparation of Molecular Dispersion Composite Particles.

A molecular dispersion is typically comprised of either micronized or unmicronized active ingredient material (e.g. SX) input material and cholesterol which are both dissolved in a common solvent and spray dried from a feedstock solution. In this case, the feedstock solution (4% w/w solids) was prepared by placing approximately 5 g of cholesterol into a 500 mL beaker. Approximately 250 mL: of acetone was added and stirred until the solids were dissolved. Approximately 5 g of salmeterol xinafoate was then added to the solution and stirred until the SX dissolved (approx 15 min). The resulting solution feedstock was then spray dried in a suitable spray dryer (e.g., Niro Mobile Minor) using the following process parameters:
Inlet Temperature—50 deg. C.
Final Outlet Temperature—35 deg. C.
Nozzle Atomization Pressure—2 bar
Feedstock feedrate—approx 22.5 mL/min
Process Gas Flow rate: 78 kg/hr
Watson Marlow Solution Pump 505 L

### Example 5

### Preparation of a 0.8% w/w Blend of Salmeterol Base In Lactose

A 20 gram blend of 5% cholesterol coated salmeterol particles was prepared with a concetration of 0.8% w/w salmeterol base in alpha lactose monohydrate. Approximately 10 grams of alpha lactose monohydrate with target particle size of 65-85μm (Borcula, The Netherlands) was weighed into the bottom of a 120 mL amber glass jar. 0.247g of 5% cholesterol coated salmeterol HNA was added on top of the lactose and 9.77 g of lactose was added on top of the mixture. The mixture was blended using a Turbula T2F blender at 96 RPM for 5 minutes. The jar was removed from the blender and excess blend was tapped from the top of the jar. One additional blending cycle was performed for 5 minutes at 96 RPM to complete the process.

### Stability of Composite Particles

Samples of particles prepared in accordance with Example 2 above were stored in controlled atmospheric conditions of 60° C. and 75% relative humidity for seven days. The percentage of active ingredient was taken at day 0 and day 7. The table below demonstrates the chemical stabilising effect that cholesterol has. The table indicates that with the active ingredient substance SX, the formulation with the active ingredient with the carrier substance in the absence of cholesterol is associated with a 7% drop in the amount of SX present in the formulation at day 7 of the test period. When cholesterol was included, the degradation was reduced to less than 2%.

It has been observed that micronized SX degrades at 60 ° C. and 75% relative humidity (RH) conditions when in a neat blend with lactose. A chemical stabilising effect of cholesterol is demonstrated as follows:

TABLE 1
The Effect of Cholesterol Coating on the Chemical Stability of Raw
Particles and Lactose Carrier Blends

% Impurities of
Composite Particles
in 0.8% Salmeterol
Base/Lactose Blend

| Target | % w/w | | 7 Days at |
|---|---|---|---|
| Cholesterol | Cholesterol | | 60° C. and |
| Content | by HPLC | Initial | 75% RH |
| MIC SX from | | 0.5 | 8.0 |
| alternate study | | | |
| Micronized SX | 0% | ND | 7.4 |
| 5% Cholesterol | 3.9% | 0.5 | 1.9 |
| Coat | | | |
| 15% Cholesterol | 13.7% | 0.4 | 1.5 |
| Coat | | | |
| 25% Cholesterol | 24.2% | 0.2 | 1.7 |
| Coat | | | |
| 50% Cholesterol | ND | 0.5 | 1.5 |
| Coat | | | |

ND = not determined

From the data below, it can be seen that the presence of a reactive environment (e.g., the presence of a lactose carrier) will yield increased impurity levels. This chemical instability may be reduced or ameliorated by incorporating cholesterol in a chemically stabilizing amount. The amount of cholesterol used is dependent on what is considered an acceptable impurity level for a particular use.

TABLE 2
Chemical Protective effect of Cholesterol on GW597901M

% Impurities of Composite
Particles in 0.4%
GW597901M Base/Lactose
Blend

| | | 7 Days at |
|---|---|---|
| Target Cholesterol | | 60° C. and |
| Content | Initial | 75% RH |
| Micronized | 3.9 | 10.0 |
| GW597901M | | |
| 2.5% Cholesterol | 4.0 | 9.3 |
| coat | | |
| 7.5% Cholesterol | 3.8 | 8.5 |
| coat | | |
| 20% Cholesterol | 3.3 | 7.4 |
| coat | | |

What is Claimed Is:

1. A method of improving the chemical stability of an active ingredient substance in a particulate formulation comprising associating the active ingredient substance with a chemically stabilising amount of cholesterol to form composite particles comprising said active ingredient substance and cholesterol.

2. The method of claim 1, wherein said active ingredient substance is in particulate form having an exterior surface and said cholesterol covers at least a portion of said exterior surface.

3. The method of claim 1, wherein is active ingredient substance comprises the group Ar—CH(OH)—CH₂—NH—R.

4. The method of claim 3, wherein said active ingredient substance comprises salmeterol, or a salt thereof.

5. The method of claim 4, wherein said active ingredient substance comprises salmeterol xinafoate.

6. The method of claim 3, wherein said active ingredient substance comprises 3-(4-{[6-({(2R)-2-hydroxy-2-[4-hydroxy-3-(hydroxymethyl)phenyl]ethyl}amino)hexyl]oxy}-butyl)benzene-sulfonamide, or a salt thereof;

7. The method of claim 7, wherein said active ingredient material is 3-(4-{[6-({(2R)-2-hydroxy-2-[4-hydroxy-3-(hydroxymethyl)phenyl]ethyl}amino)hexyl]oxy}-butyl)benzene-sulfonamide cinnamate salt.

8. The method of claim 1, wherein said active ingredient material comprises two or more therapeutic agents.

9. The method of claim 8, wherein said active ingredient material comprises a beta-agonist, and a least a further therapeutic agent selected from the group consisting comprising a beta-agonist, an anticholinergic, or a corticosteroid.

10. The method of claim 1 further comprises including one or more carriers or diluents in said particulate formulation.

11. The method of claim 12, wherein said one or more coarse carrier or diluent comprises is lactose.

12. The method of claim 1, wherein said composite particle comprises active ingredient substance in nanoparticulate form and cholesterol formed in a nanoparticulate dispersion.

13. A method of inhibiting or reducing chemical interaction between an active ingredient substance and a carrier in a solid pharmaceutical formulation, wherein the active ingredient substance is susceptible to chemical interaction with the carrier comprising the inclusion of cholesterol in said solid pharmaceutical formulation.

14. The method of claim 13 wherein the carrier is a reducing sugar.

15. The Method of claim 14 wherein the carrier is lactose.

16. The method of claim 13 wherein the cholesterol is present in an amount of from 0.1 to 90% w/w based on the total weight of the composition.

17. The method of claim 13 wherein the cholesterol is present in an amount of from 5 to 20% w/w based on the total weight of the composition.

18. The method of claim 13 wherein the active ingredient substance is present in an amount of from 0.01% to 50% w/w based on the total weight of the composition.

19. The method of claim 13 wherein the drug substance is one which includes the group Ar—CH(OH)—CH₂—NH—R.

20. The method of claim 13 wherein the solid pharmaceutical formulation is for administration by inhalation.

21. A method of treating asthma, chronic obstructive pulmonary disease (COPD), chronic or wheezy bronchitis, emphysema, respiratory tract infection, upper respiratory tract disease or rhinitis, including seasonal and allergic rhinitis comprising administration of a solid pharmaceutical formulation employing the method of claim 13.

22. A method of inhibiting or reducing chemical degradation of an active ingredient substance in a solid pharmaceutical formulation comprising the active ingredient substance and a carrier, wherein said active ingredient substance is susceptible to chemical interaction with said carrier comprising inclusion of cholesterol in said solid pharmaceutical formulation.

23. The method of claim 22 wherein the carrier is a reducing sugar.

24. The Method of claim 23 wherein the carrier is lactose.

25. The method of claim 22 wherein the cholesterol is present in an amount of from 0.1 to 90% w/w based on the total weight of the composition.

26. The method of claim 25 wherein the cholesterol is present in an amount of from 5 to 20% w/w based on the total weight of the composition.

27. The method of claim 22 wherein the active ingredient substance is present in an amount of from 0.01% to 50% w/w based on the total weight of the composition.

28. The method of claim 22 wherein the drug substance is one which includes the group Ar—CH(OH)—CH₂—NH—R.

29. The method of claim 22 wherein the solid pharmaceutical formulation is for administration by inhalation.

30. A method of treating asthma, chronic obstructive pulmonary disease (COPD), chronic or wheezy bronchitis, emphysema, respiratory tract infection, upper respiratory tract disease or rhinitis, including seasonal and allergic rhinitis comprising administration of a solid pharmaceutical formulation employing the method of claims 22.
